(19) United States
(12) Patent Application Publication
(10) Pub. No.: US 2023/0181661 A1
(43) Pub. Date: Jun. 15, 2023

(54) MUSHROOM-CONTAINING CHINESE MEDICINE COMPOUND COMPOSITION FOR KELOID SCAR TISSUE AND APPLICATION THEREOF

(71) Applicant: ASIA UNIVERSITY, TAICHUNG CITY (TW)

(72) Inventor: MING-MING LEE, Taichung City (TW)

(21) Appl. No.: 17/810,600

(22) Filed: Jul. 2, 2022

(30) Foreign Application Priority Data
Dec. 9, 2021 (TW) 110146160

(51) Int. Cl. A61K 36/06; A61K 36/9066; A61K 36/282; A61K 9/06; A61K 9/08; A61K 9/70; A61K 9/00; A61P 17/02

(52) U.S. Cl. CPC A61K 36/06; A61K 36/9066; A61K 36/282; A61K 9/06; A61K 9/08; A61K 9/7038; A61K 9/0073; A61P 17/02

(57) ABSTRACT

The present invention provides a mushroom-containing Chinese medicine compound composition for treating keloid scar tissue, the composition including: brown strain of Flammulina velutipes extract, Artemisinin, Matrine, Triptolide, Tetramethylpyrazine, Tetrandrine, Curcumin, Resveratrol, EGCG, Quercetin and Asiaticoside. Said mushroom-containing Chinese medicine compound composition is used to inhibit the proliferation of keloid fibroblast, and to treat, inhibit or reduce the symptoms of keloid fibroblast.

### CROSS-REFERENCE TO RELATED APPLICATION

This application claims the benefit of Taiwan Patent Application No. 110146160, filed on Dec. 9, 2021, in the Taiwan Intellectual Property Office, the disclosure of which is incorporated herein in its entirety by reference.

### BACKGROUND OF THE INVENTION

### 1. Field of the Invention

The present invention relates to a mushroom-containing Chinese medicine complex composition, and more particularly, to a mushroom-containing Chinese medicine complex composition for keloid scar tissue and applications thereof.

### 2. Description of the Related Art

A keloid scar is a type of scar tissue. After an injury to the skin, excessively activated fibroblasts produce excess collagen protein, thereby forming keloid scars or hypertrophic scars. The formation of keloid scar is related to a body type and a damaged site. The appearance is similar to the hypertrophic scars. However, compared to the hypertrophic scars that are mostly confined to the original injury site and may shrink, the keloid scars spread to the periphery, forms lesions that look like “crab legs”, and may not shrink over time.

Keloid scars may cause unpleasant sensations such as rubefaction, itching, tingling, tenderness or stiffness. Although most people do not experience severe pain or discomfort, the proliferation of keloid scars may still cause an aesthetic displeasure in the patient, and persistent displeasure may easily affect mental and physical conditions of the patient. Therefore, the treatment of keloid scars is still receiving widespread attention.

Currently, the major treatment options that can be considered for keloid scars include drug injections, cryotherapy, or surgical excision with respect to the lesion. Regarding drug injection, steroid drugs are mostly used. However, steroid injection may easily cause side effects such as changes in skin pigmentation, skin atrophy, and microvascular proliferation, as well as common side effects of steroid drugs such as menstrual cycle disruption. Meanwhile, the cryotherapy does not have the aforementioned drug side effects, but may still cause pain during treatment, blister or blood clot formation, permanent discoloration and pigmentation. Finally, the surgical excision may have a risk that the keloid scars recurs after surgery and becomes larger than the original lesion. Thus, it is still necessary to combine the above-described other treatment options to inhibit recurrence of keloid scars after surgery.

Therefore, there is a need in the art for more effective and less harmful treatment methods so as to inhibit the recurrence of keloid scars while reducing side effects.

### SUMMARY OF THE INVENTION

In consideration of the conventional technical problems as described above, an object of the present invention is to provide a mushroom-containing Chinese medicine complex composition to effectively treat and inhibit hypertrophic scars of keloid scars. Based on the above object, the mushroom-containing Chinese medicine complex composition of the present invention includes: 2 parts by weight of brown strain of Flammulina velutipes extract; 1 part by weight of Artemisinin; 1 part by weight of Matrine; 2 parts by weight of Triptolide; 2 parts by weight of Tetramethylpyrazine; 16 parts by weight of Tetrandrine; 4 parts by weight of Curcumin; 16 parts by weight of Resveratrol; 1 part by weight of Epigallocatechin gallate (EGCG); 2 parts by weight of Quercetin; and 2 parts by weight of Asiaticoside.

Preferably, the mushroom-containing Chinese medicine complex composition further includes 1 part by weight of Tremella fuciformis extract.

Preferably, the mushroom-containing Chinese medicine complex composition inhibits the proliferation of keloid fibroblasts, and promotes apoptosis of scar tissue, so that hypertrophic keloid scars may be treated, inhibited or reduced.

Preferably, the mushroom-containing Chinese medicine complex composition is prepared in the form of ointment, colloid, gel, solution, emulsion, patch or spray.

Based on the above object, the present invention further provides a use of the mushroom-containing Chinese medicine complex composition for preparing a drug that promotes apoptosis of scar tissue, produces tissue reconstitution, and reduces scar proliferation.

Based on the above object, the present invention further provides a use of the mushroom-containing Chinese medicine complex composition for preparing a drug that treats, inhibits or reduces keloid scars.

Next, according to the mushroom-containing Chinese medicine complex composition and the use thereof of the present invention, one or more following advantages exist:

(1) In human experiments conducted by the inventor, the mushroom-containing Chinese medicine complex composition of the present invention can remove more than 80% of hypertrophic keloid scars existing for 10 years or more, after 4 months of treatment.

(2) The mushroom-containing Chinese medicine complex composition of the present invention is composed of pure natural Chinese medicine ingredients and does not contain synthetic drugs such as steroids and has mild properties, so that the skin can be inhibited from being damaged, while side effects of steroids can also be inhibited. Therefore, the composition can be suitable for long-term use, and a scar removal effect can be remarkably realized. The therapeutic effect can maintain significant after continuous uses for more than 3 months, while general disadvantages of Western medicine, such as chemical resistance, can be inhibited, and the recurrence of keloid scars can be inhibited.

For the further understanding of the above and other aspects of the present invention, hereinafter, the exemplary embodiments will be described in detail with reference to the accompanying drawings.

### BRIEF DESCRIPTION OF THE DRAWINGS

FIGS. 1(A) to 1(C) are photographs showing the effect of a first example using a mushroom-containing Chinese medicine complex composition according to one embodiment of the present invention.

FIGS. 2(A) to 2(D) are photographs showing the effect of a second example using the mushroom-containing Chinese medicine complex composition according to one embodiment of the present invention.

FIG. 3 is a graph showing the effect of different doses (3.125, 6.25, 12.5, 50, and 100 μM) of 10 Chinese medicine ingredients according to the present invention on KFS cell growth.

FIG. 4 is a graph showing the regulation of KFS apoptosis-related proteins of the 10 ingredients of Chinese medicine according to the present invention.

### DETAILED DESCRIPTION OF THE PREFERRED EMBODIMENTS

Ointment Preparation Process of the Present Invention

When calculated by the dry weight ratio of a material, the ointment includes: 1 part by weight of Tremella fuciformis extract; 2 parts by weight of brown strain of Flammulina velutipes extract; 1 part by weight of Artemisinin; 1 part by weight of Matrine; 2 parts by weight of Triptolide; 2 parts by weight of Tetramethylpyrazine; 16 parts by weight of Tetrandrine; 4 parts by weight of Curcumin; 16 parts by weight of Resveratrol; 1 part by weight of Epigallocatechin gallate (EGCG); 2 parts by weight of Quercetin; and 2 parts by weight of Asiaticoside.

After extracting the mushroom extract with hot water at 100° C. (weight ratio 1:1), the above 10 Chinese herbal ingredients are added and mixed with an ointment base, thereby preparing an ointment formulation.

Results on Example Implementation

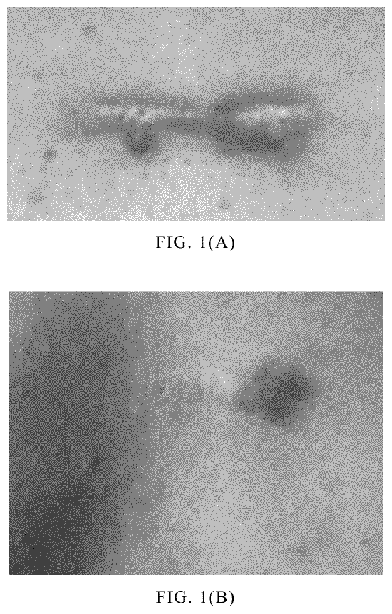

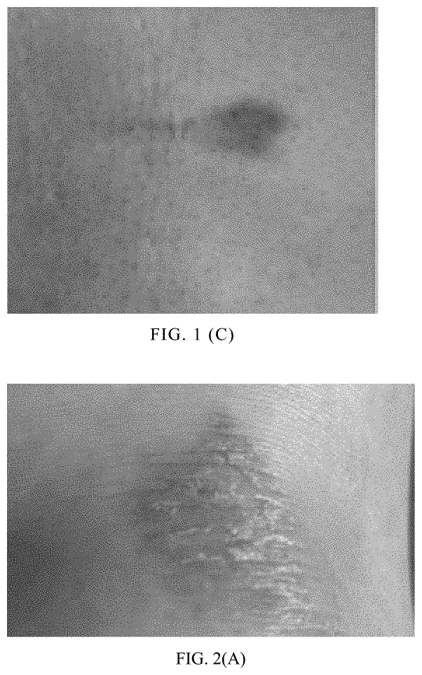

The example is provided as follows, in which the ointment is directly applied to the affected area. As shown in FIG. 1(A), the first example of a keloid scar is a scar having been more than 10 years, and has problems that affect the quality of life, such as pain, itching, and foreign body sensation. Medication and surgical treatment have been conducted, but failed to diminish the scar. Instead, a new scar on the right side in FIG. 1(A) has derived from the original scar on the left side, which is a typical feature of keloid scars.

After 1 month of treatment using the ointment prepared according to the present invention, as shown in FIG. 1(B), the flexibility, size, thickness, area, and symptom such as pain, itchiness, and foreign body sensation were significantly improved. As shown in FIG. 1(C), more than 90% of the scar area disappeared within 3 months of use.

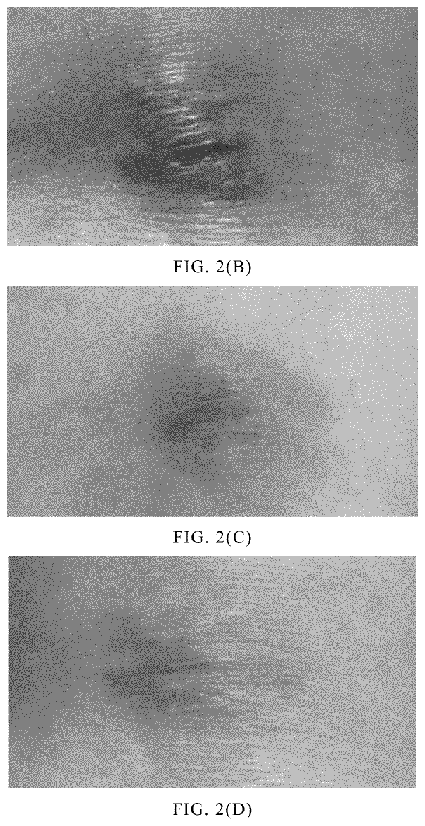

The second example as shown in FIG. 2(A) is an example of a typical bruise scar. The scar was rarely cured because the wound scabs and peels off repeatedly for 2 weeks. Moreover, disinfection of the affected area with iodine causes pigmentation. After one week of treatment using the ointment prepared according to the present invention, the lesion was cured but the scar still remained as shown in FIG. 2(B); After 3 weeks of use, it can be clearly seen that more than 90% of the scar is removed as shown in FIG. 2(C); After 4 weeks of use, it can be seen that pigmentation is remarkably removed as shown in FIG. 2(D).

Experimental Method and Results of the Chinese Medicine Ingredients of the Present Invention in Keloid Cells

Materials

Fetal bovine serum (FBS), penicillin G, Dulbecco's modified eagle medium (DMEM), and streptomycin prepared by Invitrogen (Carlsbad, Calif., USA). Pyruvic acid and non-essential amino acids prepared by Biological Industries (Kibbutz Beit Haemek, Israel). Primary antibody prepared by Santa Cruz Biotechnology (Santa Cruz, Calif., USA) against procaspase 3/8/9, cleaved caspase 3/8/9, cleaved PARP, cell pigment c, bax, bcl-2 and β-actin. Secondary antibody prepared by Santa Cruz Biotechnology coupled to horseradish peroxidase (HRP). Other reagents prepared by Sigma-Aldrich (St. Louis, Mo., USA).

Preparation of Test Formulations

The combination of 10 Chinese medicine ingredients in this experiment is shown in Table 1 below, and used as an in vitro study.

TABLE 1

| | Mixed dose | | | | | | | | | |
|---|---|---|---|---|---|---|---|---|---|---|
| | No. | | | | | | | | | |
| | 1 | 2 | 3 | 4 | 5 | 6 | 7 | | | 10 |
| | Arte- | Ma- | Tri- | Tetram- | Te- | Cur- | Re- | | 9 | Asia- |
| Ingre- | mis- | tri- | pto- | ethyl- | tran- | cu- | svera- | 8 | Quer- | tico- |
| dients | inin | ne | lide | pyrazine | drin | min | trol | EGCG | cetin | side |
| uM | 3.125 | 3.125 | 6.25 | 6.25 | 50 | 12.5 | 50 | 3.125 | 6.25 | 6.25 |
| Ratio | 1 | 1 | 2 | 2 | 16 | 4 | 16 | 1 | 2 | 1 |

Experiment Result

Cell Culture

KFS cells were obtained from the Biosource Collection and Research Center (BCRC) of the Food Industry Research and Development Institute (Hsinchu, Taiwan). KFS cells were cultured in Dulbecco's modified eagle medium (DMEM) containing 10% FBS, 1.5 g/L sodium bicarbonate, 4 mM L-glutamine, 4.5 g/L glucose, 100 units/mL penicillin G, and 100 μg/mL streptomycin sulfate. All cells were incubated in a humidified environment with 5% CO2 and 37° C. The medium was refreshed every 2 days.

Cell Metabolic Activity Assay (MTT Assay)

KFS cells (2×10⁴ cells/well) were seeded in 96-well plates overnight. KFS cells were exposed to different concentrations of a test drug in 100 μL of medium (for example, combination of 10 Chinese medicine ingredients). After 24 hours treatment, 10 μL of 5 mg/mL MTT (3-(4,5-dimethylthiazol-2-yl)-2,5-diphenyltetrazolium bromide) was added to each well. After incubation for 4 hours, cells were washed twice with 1×PBS. Next, 200 μL of dimethyl sulfoxide (DMSO) was added to each well. An absorbance value at 570 nm was determined for each well by using 650 nm as a reference wavelength. Compared with a control group (untreated with reagents), the absorbance may be correlated with the percentage of active cells. The cell metabolic activity rate is calculated as follows: Cell metabolic activity (%)=OD (treatment)/OD (control)×100%.

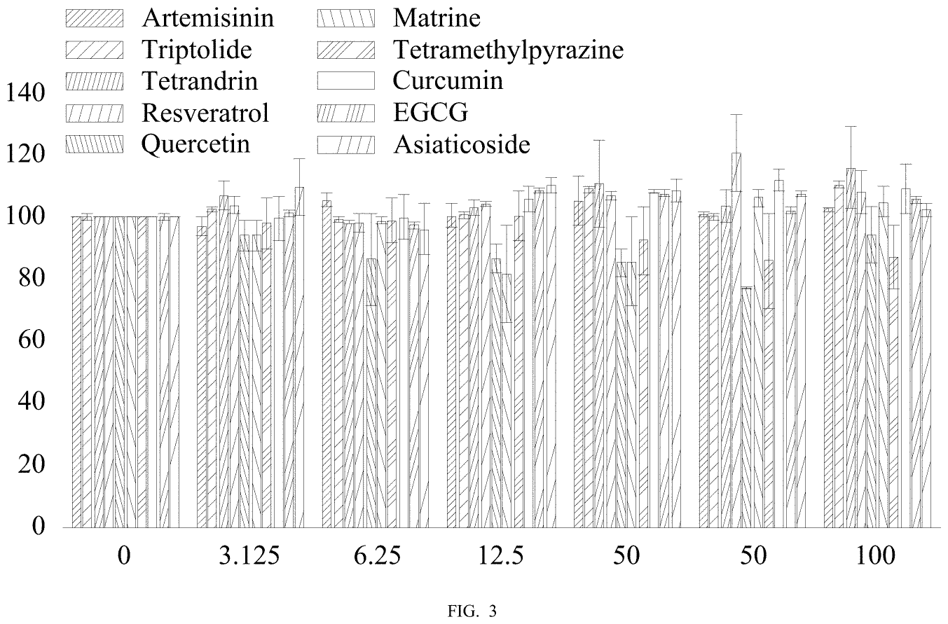

The results are shown in FIG. 3. The combination of 10 Chinese medicine ingredients did not affect the cell metabolic activity of KFS cells. FIG. 3 shows the effect of different doses of 10 compounds (3.125, 6.25, 12.5, 50 and 100 μM) on KFS cell growth. That is, after 24 hours of treatment, all IC50 values of the 10 traditional Chinese medicine components were >100 μM.

Western Blot Analysis

The obtained cells were cleaved with a PRO-PREP protein extraction reagent kit (iNtRON Biotechnology, Gyeonggi-do, Korea), and centrifuge with 10,000 rpm for 30 minutes at 4° C. A supernatant was incubated in 6× loading buffer containing 0.35 M Tris-HCl (pH 6.8), 10% SDS, 30% glycerol, 0.12% bromophenol blue, and 6% β-mercaptoethanol at 95° C. for 5 minutes. About 50 μg of total protein was isolated through 10% sodium dodecyl sulfate-polyacrylamide gel electrophoresis (SDS-PAGE), and then, blotted onto a polyvinylidene fluoride (PVDF) membrane (NEF1002001PK; PerkinElmer, Boston, Mass., USA). A non-specific binding of the blotting membrane was blocked using 5% skim milk dissolved in 1× Tris buffered saline (TBS) and 0.2% Tween20 (0.2% TBST). The membrane was incubated overnight at 4° C. with primary antibody (in 0.2% TBST). Then, secondary antibody (in 0.2% TBST) was applied at 4° C. for 3 hours. The predicted protein bands may be visualized by using an ECL reaction (Amersham, Arlington Height, Ill., USA). Luminescence signals were acquired using a Fujifilm LAS-4000 system (SanLeandro, Calif., USA). A band intensity was analyzed using Multi Gauge software (Fujifilm). The band intensity of individual protein was normalized to that of β-actin, and expressed as fold change compared to the control group (untreated with reagent).

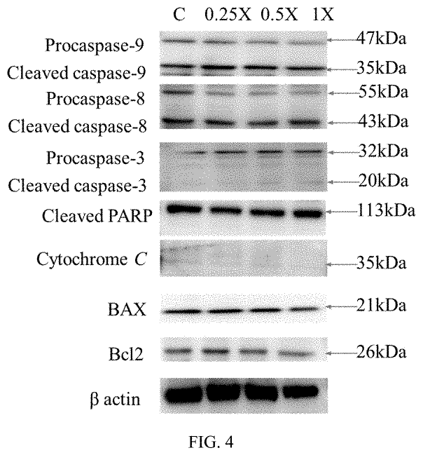

FIG. 4 shows the regulation of KFS apoptosis-related protein by the combination of 10 Chinese medicine ingredients. The 10 Chinese medicine ingredients were administered to KFS cells via the combination of 0.25×, 0.5× and 1.0×.

### CONCLUSION

According to the cell test method, after dissolving the 10 Chinese medicine ingredients (1. Artemisinin, 2. Matrine, 3. Triptolide, 4. Tetramethylpyrazine, 5. Tetrandrine, 6. Curcumin, 7. Resveratrol, 8. EGCG, 9. Quercetin, 10. Asiaticoside) in an appropriate solution through the method described above, the effects on the proliferation of keloid fibroblasts and the expression of apoptosis proteins were tested. It is confirmed that Tetrandrine, Curcumin, and Resveratrol thereamong inhibit the growth of keloid fibroblasts when the cell concentration is 6.25 to 100 micromolar concentration.

Next, the 10 Chinese medicine ingredients (1. Artemisinin, 2. Matrine, 3. Triptolide, 4. Tetramethylpyrazine, 5. Tetrandrine, 6. Curcumin, 7. Resveratrol, 8. EGCG, 9. Quercetin, 10. Asiaticoside) were mixed in the following ratio, so as to be acted on keloid fibroblasts at a concentration ratio of 0.25 times to 1 times. It was found that the protein expression of the anti-apoptosis protein Bcl2 may be reduced. For the caspase protease family that produces apoptosis, the combination of the 10 Chinese medicine ingredients may reduce the expression of procaspase 9 in the endogenous pathway of apoptosis and the procaspase 8 protein in the exogenous pathway of apoptosis. The response to increases in cleaved caspase 9 and cleaved caspase 8 signifies that the response pathway for apoptosis of keloid fibroblasts is enhanced.

The above examples are merely some embodiments of the mushroom-containing Chinese medicine complex composition for keloid scar tissue of the present invention, and are not intended to limit the scope of the invention. Simple equivalent changes and modifications of the spirit or relative components according to the embodiments disclosed in the claims and the detailed description for the Chinese medicine complex composition of the present invention will be understood as covered within the scope of the invention.

What is claimed is:

1. A mushroom-containing Chinese medicine complex composition for keloid scar tissue, the mushroom-containing Chinese medicine complex composition comprising:
2 parts by weight of brown strain of Flammulina velutipes extract; 1 part by weight of Artemisinin; 1 part by weight of Matrine; 2 parts by weight of Triptolide; 2 parts by weight of Tetramethylpyrazine; 16 parts by weight of Tetrandrine; 4 parts by weight of Curcumin; 16 parts by weight of Resveratrol; 1 part by weight of Epigallocatechin gallate (EGCG); 2 parts by weight of Quercetin; and 2 parts by weight of Asiaticoside.

2. The mushroom-containing Chinese medicine complex composition of claim 1, further comprising:
1 part by weight of Tremella fuciformis extract.

3. The mushroom-containing Chinese medicine complex composition of claim 1, wherein the mushroom-containing Chinese medicine complex composition is configured such that a proliferation of keloid fibroblasts is inhibited and an apoptosis of scar tissue cells is promoted, so that hypertrophic scars of keloid scars are treated, inhibited or reduced.

4. The mushroom-containing Chinese medicine complex composition of claim 2, wherein the mushroom-containing Chinese medicine complex composition is configured such that a proliferation of keloid fibroblasts is inhibited and an apoptosis of scar tissue cells is promoted, so that hypertrophic scars of keloid scars are treated, inhibited or reduced.

5. The mushroom-containing Chinese medicine complex composition of claim 1, wherein the mushroom-containing Chinese medicine complex composition is prepared in a form of ointment, colloid, gel, solution, emulsion, patch or spray.

6. The mushroom-containing Chinese medicine complex composition of claim 2, wherein the mushroom-containing Chinese medicine complex composition is prepared in a form of ointment, colloid, gel, solution, emulsion, patch or spray.

7. A use of the mushroom-containing Chinese medicine complex composition according to claim 1 for preparing a drug that promotes apoptosis of scar tissue, produces tissue reconstitution, and reduces scar proliferation.

8. A use of the mushroom-containing Chinese medicine complex composition according to claim 2 for preparing a drug that promotes apoptosis of scar tissue, produces tissue reconstitution, and reduces scar proliferation.

9. A use of the mushroom-containing Chinese medicine complex composition according to claim 1 for preparing a drug for treating, inhibiting or reducing keloid scars.

10. A use of the mushroom-containing Chinese medicine complex composition according to claim 2 for preparing a drug for treating, inhibiting or reducing keloid scars.
